(19) United States
(12) Patent Application Publication
(10) Pub. No.: US 2023/0281815 A1
(43) Pub. Date: Sep. 7, 2023

(54) CERVICAL CANCER SCREENING SUPPORT SYSTEM, CERVICAL CANCER SCREENING SUPPORT METHOD, RECORDING MEDIUM CARRYING CERVICAL CANCER SCREENING SUPPORT PROGRAM, AND SMARTPHONE BUILT WITH SMARTPHONE APPLICATION CARRYING CERVICAL CANCER SCREENING SUPPORT PROGRAM

(71) Applicants: FUTURE UNIVERSITY HAKODATE, Hakodate-shi (JP); SAPPORO MEDICAL UNIVERSITY, Sapporo-shi (JP)

(72) Inventors: Yuichi FUJINO, Hakodate-shi (JP); Ikuma SATO, Hakodate-shi (JP); Yuta NAMBU, Hakodate-shi (JP); Tasuku MARIYA, Sapporo-shi (JP); Shota SHINKAI, Sapporo-shi (JP); Hiroko ASANUMA, Sapporo-shi (JP)

(21) Appl. No.: 18/318,394

(22) Filed: May 16, 2023

Related U.S. Application Data
(63) Continuation of application No. PCT/JP2021/042014, filed on Nov. 16, 2021.

(30) Foreign Application Priority Data
Nov. 16, 2020 (JP) 2020-190423

(51) Int. Cl. G06T 7/00; G06V 20/69; G06V 10/774; G06T 7/70; G16H 10/40; C12M 1/34

(52) U.S. Cl. CPC G06T 7/0012; G06V 20/698; G06V 20/695; G06V 10/774; G06T 7/70; G16H 10/40; C12M 41/36; G06V 2201/03; G06T 2207/30024; G06T 2207/20081; G06T 2207/20076; G06V 10/82

(57) ABSTRACT

A cervical cancer screening support system includes: an image acquisition unit that acquires a micrograph of a cell for cytodiagnosis of a cervix of uterus; a cell aggregate recognition unit that recognizes a cell aggregate in the micrograph; and an output unit that outputs a class applicable to a cell belonging to the cell aggregate.

### BACKGROUND OF THE INVENTION

### 1. Field of the Invention

The present invention relates to a cervical cancer screening support system, a cervical cancer screening support method, a cervical cancer screening support program and smartphone application.

### 2. Description of the Related Art

In cytodiagnosis for uterine cervical cancer screening, a technology is proposed that realizes screening such as automatic detection and discrimination of cellular findings by using a general-purpose object detection scheme based on deep learning (see, for example, patent literature 1).
[Non-patent literature 1] Jith, O. U. N.; Harinarayanan, K. K.; Gautam, S.; Bhaysar, A.; Sao, A. K. DeepCerv: Deep Neural Network for Segmentation Free Robust Cervical Cell 25 Classification. In Computational Pathology and Ophthalmic Medical Image Analysis; Lecture Notes in Computer Science; Springer: Berlin, Germany, 2018; pp. 86.94.
[Non-patent literature 2] Jith, O. U. N.; Harinarayanan, K. K.; Gautam, S.; Bhaysar, A.; Sao, A. K. DeepCerv: Deep Neural Network for Segmentation Free Robust Cervical Cell Classification. In Computational Pathology and Ophthalmic Medical Image Analysis; Lecture Notes in Computer Science; Springer: Berlin, Germany, 2018; pp. 86.94.
[Non-patent literature 3] K. Bora, M. Chowdhury, L. B. Mahanta, M. K. Kundu and A. K. Das, “Pap Smear Image Classification Using Convolutional Neural Network”, Tenth Indian Conference on Computer Vision, Graphics and Image Processing, 2016.
[Non-patent literature 4] ResNeSt: Split-Attention Networks. 2020, http://arxiv.org/abs/2004.08955.
[Non-patent literature 5] Saso Dzeroski, Bernard Zenko, Machine Learning, 54, 255-273, 2004

The scheme described in non-patent literature 1 enables detection of cellular findings and classification according to malignancy in a multiple-cell cytodiagnostic image (an image in which multiple cells are found). However, this scheme has an average recall factor less than 70% and so is expected to increase overlooking of positive findings, i.e., false negative findings. In essence, a general-purpose object detection scheme like this is useful when applied to cytodiagnosis but cannot be said to be sufficient as a screening scheme due to its low precision.

### SUMMARY OF THE INVENTION

The present invention addresses the issue, and a purpose thereof is to provide a highly precise screening scheme for cytodiagnosis for uterine cervical cancer screening.

A cervical cancer screening support system according to an aspect of the present invention includes: an image acquisition unit that acquires a micrograph of a cellular specimen for cytodiagnosis of a cervix of uterus; a cell aggregate recognition unit that recognizes a cell aggregate in the micrograph for atypia classification based on a cell aggregate in the cellular specimen; and an output unit that outputs a class, including an atypia, applicable to a cell belonging to the cell aggregate.

In the cervical cancer screening support system, an area including the cell aggregate may include a background around the cell aggregate.

In the cervical cancer screening support system, the cell aggregate recognition unit may recognize a cell aggregate by using YOLO algorithm.

In the cervical cancer screening support system, the cell aggregate recognition unit may recognize a cell aggregate in real time.

The cervical cancer screening support system may further include an estimation unit that estimates and outputs, when a micrograph acquired by the image acquisition unit is input, a position of a cell found to be likely to be abnormal in the micrograph and a class applicable to the cell likely to be abnormal, by using an estimation model generated through machine learning according to an object detection algorithm, using, as training data, a marked cell aggregate, of cell aggregates recognized by the cell aggregate recognition unit, that includes an atypical cell and an atypia of a cell included in the marked cell aggregate.

Another aspect of the present invention also relates to a cervical cancer screening support system. The system includes: an image acquisition unit that acquires a micrograph of a cell collected from a cervix of uterus; a first estimation unit that estimates and outputs, when the micrograph acquired by the image acquisition unit is input, a position of a cell found to be likely to be abnormal in the micrograph and a class applicable to the cell likely to be abnormal, by using a first estimation model generated through machine learning according to an object detection algorithm, using, as training data, the micrograph, the position of the cell found to be likely to be abnormal in the micrograph, and the class applicable to the cell likely to be abnormal, the class being a result of examination for classification; and an image conversion unit that extracts from the micrograph an image of each cell located at the position estimated by the first estimation unit and converts each extracted image into a post-conversion image of a predetermined format; and a second estimation model that estimates and outputs, when the post-conversion image is input, a probability that the cell in the post-conversion image fits into each of the classes by using a second estimation model generated through machine learning according to an image classification algorithm, using, as training data, the image of the cell likely to be abnormal and the probability that the cell likely to be abnormal fits into each of the classes.

The class may be a class of Bethesda classification.

The object detection algorithm may be YOLO algorithm.

The image classification algorithm may be a convolutional neural network.

The cervical cancer screening support system may further include: a third estimation model generated by integrating the class estimated by the first estimation model as being applicable to the cell likely to be abnormal and the probability, estimated by the second estimation model, that the cell in the post-conversion image fits into each of the classes, the third estimation model estimating and outputting the probability that the cell likely to be abnormal fits into each of the classes.

The third estimation model may be generated by using stacking ensemble learning.

The cervical cancer screening support system may include: a smartphone including the image acquisition unit; and a data processing apparatus connected to the smartphone via a network and including the first estimation unit, the image conversion unit, and the second estimation unit.

An external apparatus may be adapted to be connected to the data processing apparatus.

The data processing apparatus may include a database that stores the position of the cell likely to be abnormal and the class applicable to the cell likely to be abnormal estimated by the first estimation unit, and the probability, estimated by the second estimation unit, that the cell in the post-conversion image fits into each of the classes.

The cervical cancer screening support system may be connected via a network to an external network including the aforementioned cervical cancer screening support system, wherein the database may store the position of the cell likely to be abnormal and the class applicable to the cell likely to be abnormal estimated by the first estimation unit of the external system, and the probability, estimated by the second estimation unit of the external system, that the cell in the post-conversion image fits into each of the classes.

The cervical cancer screening support system may include the above-described cell aggregate recognition unit.

Another aspect of the present invention relates to a cervical cancer screening support system. The method includes: acquiring, by using an image acquisition unit, a micrograph of a cell collected from a cervix of uterus; recognizing a cell aggregate in the micrograph; and outputting a class applicable to a cell belonging to the cell aggregate.

Another aspect of the present invention also relates to a cervical cancer screening support method. The method includes: acquiring, by using an image acquisition unit, a micrograph of a cell collected from a cervix of uterus; a first estimation estimating, when the micrograph acquired by the image acquisition unit is input, a position of a cell found to be likely to be abnormal in the micrograph and a class applicable to the cell likely to be abnormal, by using a first estimation model generated through machine learning according to an object detection algorithm, using, as training data, the micrograph, the position of the cell found to be likely to be abnormal in the micrograph, and the class applicable to the cell likely to be abnormal, the class being a result of examination for classification; extracting from the micrograph an image of each cell located at the position estimated by the first estimation and converting each extracted image into a post-conversion image of a predetermined format; and a second estimation estimating, when the post-conversion image is input, a probability that the cell in the post-conversion image fits into each of the classes by using a second estimation model generated through machine learning according to an image classification algorithm, using, as training data, the image of the cell likely to be abnormal and the probability that the cell likely to be abnormal fits into each of the classes.

Another aspect of the present invention also relates to a cervical cancer screening support program. The program causes a computer to execute a method comprising: acquiring, by using an image acquisition unit, a micrograph of a cell collected from a cervix of uterus; recognizing a cell aggregate in the micrograph; and outputting a class applicable to a cell belonging to the cell aggregate.

Another aspect of the present invention also relates to a cervical cancer screening support program. The program causes a computer to execute a method comprising: acquiring, by using an image acquisition unit, a micrograph of a cell collected from a cervix of uterus; a first estimation estimating, when the micrograph acquired by the image acquisition unit is input, a position of a cell found to be likely to be abnormal in the micrograph and a class applicable to the cell likely to be abnormal, by using a first estimation model generated through machine learning according to an object detection algorithm, using, as training data, the micrograph, the position of the cell found to be likely to be abnormal in the micrograph, and the class applicable to the cell likely to be abnormal, the class being a result of examination for classification; extracting from the micrograph an image of each cell located at the position estimated by the first estimation and converting each extracted image into a post-conversion image of a predetermined format; and a second estimation estimating, when the post-conversion image is input, a probability that the cell in the post-conversion image fits into each of the classes by using a second estimation model generated through machine learning according to an image classification algorithm, using, as training data, the image of the cell likely to be abnormal and the probability that the cell likely to be abnormal fits into each of the classes.

Another aspect of the present invention relates to a smartphone application. The smartphone application includes the aforementioned cervical cancer screening support program.

Optional combinations of the aforementioned constituting elements, and mutual substitution of constituting elements and implementations of the present invention between methods, apparatuses, programs, transitory or non-transitory recording mediums carrying the program, systems, etc. may also be practiced as additional modes of the present invention.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 is a functional block diagram of a cervical cancer screening support system according to the first embodiment;

FIG. 2 is a functional block diagram of a cervical cancer screening support system according to the second embodiment;

FIG. 3 schematically shows an exemplary configuration of the cervical cancer screening support system according to the first embodiment;

FIG. 4 schematically shows another exemplary configuration of the cervical cancer screening support system according to the first embodiment;

FIG. 5 is a flowchart showing steps in a cervical cancer screening support method according to the third embodiment;

FIG. 6 schematically shows an embodiment of the smartphone application according to the fifth embodiment;

FIG. 7 is a functional block diagram of a cervical cancer screening support system according to the sixth embodiment;

FIG. 8 is a functional block diagram of a cervical cancer screening support system according to the seventh embodiment; and

FIG. 9 is a flowchart showing steps in a cervical cancer screening support method according to the eighth embodiment.

### DETAILED DESCRIPTION OF THE INVENTION

The invention will now be described by reference to the preferred embodiments. This does not intend to limit the scope of the present invention, but to exemplify the invention.

Hereinafter, the invention will be described based on preferred embodiments with reference to drawings. In the embodiments and variations, the same or equivalent constituting elements and components shall be denoted by the same reference numerals, and duplicative explanations will be omitted appropriately. The dimension of components in the drawings shall be enlarged or reduced as appropriate to facilitate understanding. Those of the components that are not material to the description of the embodiments are omitted in the drawings. Terms including ordinal numbers (first, second, etc.) are used to explain various constituting elements, but the terms are used merely for the purpose of distinguishing one constituting element from the other constituting elements and shall not construed as limiting the constituting elements.

Basic knowledge will be given before describing specific embodiments. Cervical cancer is most common among gynecological malignant tumors. In Japan, it ranks No. 1 as the cancer affecting women of 0-49 years old in terms of decease rate and No. 3 in terms of death rate. Currently, hysterectomy is the main stream treatment of cervical cancer from the perspective of giving top priority to life extension. When a cervical cancer is discovered at an early state, it is possible to conserve the uterus by a treatment such as partial conization. When tumor invasion is found, however, total hysterectomy will be necessary. When metastasis to another site is found, further, radiation treatment or anticancer drug treatment will be necessary. In this background, early discovery in cervical cancer screening is called for.

Cervical cancer screening is generally performed in two-stage decision process called cytoscreening, which is based on brushing cytology. In a decision process in the first stage (hereinafter, “primary screening”), a cytotechnologist observes cells collected by brushing the cervix of uterus under a microscope and makes a presumptive diagnosis to see whether any of the following Bethesda classification classes is applicable. Then, in a decision process in the second stage (hereinafter, “secondary screening”) the cytotechnologist makes a final decision based on microscopic observation and the result of aforementioned presumptive diagnosis.

Bethesda classification is one method of presenting cytodiagnostic results and classifies cytodiagnostic results as follows according to malignancy (the classification pertains to squamous epithelial cells).

NILM (negative)

ASC-US (cannot exclude low-grade squamous intraepithelial lesion)

LSIL (low-grade squamous intraepithelial lesion)

ASC-H (cannot exclude high-grade squamous intraepithelial lesion)

HSIL (high-grade squamous intraepithelial lesion)

SCC (cannot exclude squamous cell carcinoma)

NILM patients are determined to have no abnormalities. ASC-US patients need detailed examination such as HPV test. LSIL-SCC patients need detailed examination such as colposcopic-directed biopsy.

Cytoscreening has a problem in that specialist doctors and cytotechnologists are heavily loaded. In other words, advanced diagnosis that maximizes sensitivity to prevent cancer patients from being overlooked and, at the same time, maximizes specificity to prevent healthy subjects from being found positive. It is also required to increase the speed of diagnosis to address an increase in the number of patients diagnosed in association with an increase in opportunities for group cervical cancer examination recently. These requirements increase loads on specialist doctors and cytotechnologists more and more.

Further, cytoscreening has a problem in that a criterion that allows diagnosing in a lucid and mechanical manner is not defined. In cytoscreening, a feature amount that carries weight is defined, but then much vagueness remains in respect of a quantitative threshold value to enable a determination that cells are actually abnormal based on the feature amount. For this reason, diagnosis capability would depend on the experience of those carrying out the examination, and the diagnosis result varies depending on the hospital where the examination is carried out and who carries out the examination.

Products such as “FocalPoint” (registered trademark) from Becton Dickinson and “ThinPrep” from HOLOGIC are known as systems for supporting cytoscreening work. These products recognize the size and color strength of nucleuses of cells and present multiple fields of view in which abnormal cells are likely to be found but are not concerned with diagnosis at all. In essence, these products can present an image that includes a suspected portion but cannot show what kind of disease is suspected. In this respect, these products fall short of resolving the aforementioned problem. An additional problem is that these products are quite expensive in terms of both initial cost and running cost.

It is expected to reduce loads on special doctors, etc. and enable diagnosis that does not vary, by realizing automatic screening using information processing technology. A large number of schemes for classifying a single-cell cytodiagnostic image (an image in which one cell is found) have been proposed in the related art directed to automatic cytodiagnostic image classification. Two-class (normal/abnormal) classification schemes (e.g., non-patent literature 2) and multi-class classification schemes configured to discriminate malignancy (e.g., non-patent literature 3) are available, and both realize highly precise classification. However, these schemes target single-cell cytodiagnostic images, namely, images with a relatively small noise, and so are designed on a premise that single-cell characteristics can be acquired suitably. Therefore, these schemes cannot be directly applied to images populated with multiple cells and used in cytoscreening scenes.

Similarly, two-class (normal/abnormal) classification schemes (e.g., non-patent literature 4) and multi-class classification schemes configured to discriminate malignancy (e.g., non-patent literature 1) are available for classification targeting multi-cell cytodiagnostic images. A majority of two-class classification schemes isolate a multi-cell cytodiagnostic image into single-cell cytodiagnostic images by using segmentation or deep learning. The isolated single-cell cytodiagnostic image is subject to classification using existent single-cell characteristics. The precision in this case is poorer as compared with single-cell cytodiagnostic image classification due to insufficient precision in the single-cell isolation process, but a precision of 95% or higher is reported.

The precision of multiclass classification schemes in response to a multiple-cell cytodiagnostic image cannot be said to be sufficient. Schemes of detecting cellular findings in a multiple-cell cytodiagnostic image that use a general-purpose object detection scheme are proposed (see, for example, patent literature 1). In these schemes, pre-processes such as isolation into singular cells, artifact removal, and segmentation can be omitted by using a general-purpose object detection scheme. Moreover, these schemes can be said to meet requirements for cytoscreening more satisfactorily by making detection of cellular findings and malignancy classification in a target multiple-cell cytodiagnostic image possible. However, these schemes have an average recall factor of less than 70% and so is expected to increase overlooking of positive finding, i.e., false negative finding. In essence, a general-purpose object detection scheme like this is useful when applied to cytoscreening but does not fully meet the requirement as a screening scheme due to its low precision.

### First Embodiment

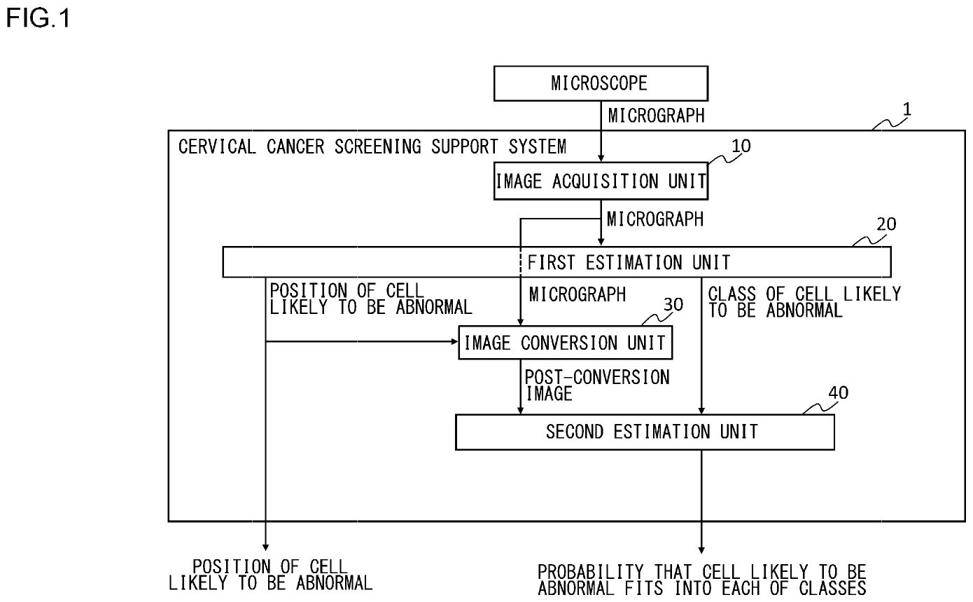

FIG. 1 is a functional block diagram of a cervical cancer screening support system 1 according to the first embodiment. The cervical cancer screening support system 1 is provided with an image acquisition unit 10, a first estimation unit 20, an image conversion unit 30, and a second estimation unit 40.

The image acquisition unit 10 acquires a micrograph of cells collected from the cervix of uterus. These cells are usually collected by scraping an examined portion by a brush, a paddle, or the like. The image acquisition unit 10 is an arbitrary camera such as a microscope camera, a camera on a smartphone, and a commercial digital camera attached to a microscope by using an adaptor. The image acquisition unit 10 inputs image data for the acquired micrograph to the first estimation unit 20 and the image conversion unit 30.

When the image data for the micrograph acquired by the image acquisition unit 10 is input, the first estimation unit 20 uses the first estimation model to estimate and output the position of cell found to be likely to be abnormal in the micrograph and the class of the cell likely to be abnormal (i.e., which of predetermined classes that each of the cells likely to be abnormal fits into, the class being a result of examination for classification). For example, a case is considered where the first estimation model determines that there are 10 cells likely to be abnormal in a micrograph and estimates the positions of these 10 cells. In this process, the first estimation unit 20 calculates, as position information on each of the 10 cells, the x coordinate, y coordinate, height, and width of a rectangular area in which the cell is included in a floating decimal mode. In this process, the first estimation model estimates which of ASC-US, LSIL, ASC-H, HSIL, and SCC of Bethesda classification mentioned above is applicable to each of the 10 cells (NILM means “not abnormal” and so is excluded). The first estimation unit 20 calculates, as class information for each of the 10 cells, an integer value for discriminating a Bethesda class applicable to the cell. Thus, the first estimation unit 20 outputs the position of the cell likely to be abnormal and the class of the cell likely to be abnormal to the second estimation unit 40.

The first estimation model is generated through machine learning according to an object detection algorithm, using, as training data, the micrograph, the position of cell found to be likely to be abnormal in the micrograph, and the class applicable to each of the cells likely to be abnormal. In the case of a cell likely to be abnormal such as a tumor, the characteristics thereof such as shape, size, color, chromatic value, gray scale, texture, and inversion identified in the image are different from those of healthy cells. These characteristics identified in the image can be highlighted depending on the case by subjecting raw image data to a process such as image inversion (horizontal and vertical), blurring, noise cancellation, gamma correction, and filtering. It is therefore possible to estimate the position of cell likely to be abnormal and the class applicable thereto with high precision, by using the micrograph including these features as training data and using an appropriate object detection algorithm. It is preferable to use, for example, YOLO algorithm as a machine learning algorithm for generating the first estimation model from the perspective of balance between detection speed and detection precision. However, the embodiment is not limited to this, and an arbitrary object detection algorithm may be used.

The image conversion unit 30 first extracts an image of each cell located at the position estimated by the first estimation unit from the micrograph. As described above, the first estimation model estimates the positions of 10 cells likely to be abnormal so that the image conversion unit 30 extracts images of cells at these 10 positions. The image conversion unit 30 then converts the extracted images into post-conversion images of a predetermined format. A description will be given of this. The images of the 10 cells extracted as described above are not necessarily suitable as images that should be input to the second estimation unit 40. In other words, the format of these 10 extracted images does not necessarily match the format of the second estimation unit 40. Accordingly, the image conversion unit 30 converts the 10 images described above into images (hereinafter, referred to as “post-conversion images”) adapted to the input format of the second estimation unit 40. More specifically, the image conversion unit 30 converts the shape, size, aspect ratio, pixel count, etc. of each extracted image into those of a post-conversion image adapted to the input format of the second estimation unit 40. The image conversion unit 30 inputs the post-conversion image thus obtained to the second estimation unit 40.

When the post-conversion image resulting from the conversion by the image conversion unit 30 is input, the second estimation unit 40 uses the second estimation model to estimate and output a probability that the cell in the post-conversion image fits into each of the classes. To follow the example described above, a case where 10 post-conversion images are input will be considered. The second estimation unit 40 makes an estimation such that the probability that the cell in the first post-conversion image fits into ACS-US of Bethesda classification is XA %, the probability that LSIL is applicable is XL %, . . . , the probability that SCC is applicable is XS %, the probability that the cell in the second post-conversion image fits into ACS-US is YA %, . . . , the probability that the cell in the tenth post-conversion image fits into ACS-US is ZS %, etc. In this way, the second estimation unit 40 calculates a matrix of 10 rows and 5 columns as information on an estimated probability that each of the 10 cells fits into the 5 classes of Bethesda classification. Thus, the second estimation unit 40 outputs a probability that the cell likely to be abnormal fits into each of the classes.

The second estimation model is generated through machine learning according to an image classification algorithm, using, as training data, the image of the cell likely to be abnormal and the probability that the cell likely to be abnormal fits into each of the classes. In the case of cells likely to be abnormal such as a tumor, as described above, the characteristics thereof such as shape, size, color, chromatic value, gray scale, and texture identified in the image vary depending on malignancy. These characteristics identified in the image can be highlighted depending on the case by subjecting raw image data to a process such as image inversion (horizontal and vertical), blurring, noise cancellation, gamma correction, and filtering. It is therefore possible to estimate the probability that the cell likely to be abnormal fits into each of the classes with high precision, by using the micrograph including these features as training data and using an appropriate image classification algorithm. It is preferable to use, for example, a convolutional neural network as a machine learning algorithm for generating the second estimation model from the perspective of excellent image classification capabilities. However, the embodiment is not limited to this, and an arbitrary image classification algorithm may be used.

According to this embodiment, it is possible to estimate the position of a cell likely to be abnormal and the probability that the cell likely to be abnormal fits into each of the classes with high precision in cytodiagnosis for uterine cervical cancer screening. This improves precision of diagnosis and reduces loads on specialist doctors and cytotechnologists.

### Second Embodiment

In the embodiment described above, the first estimation model is used to estimate the position of the cell likely to be abnormal and the class of the cell likely to be abnormal.

The second estimation model is used to estimate the probability that the cell in the post-conversion image (the cell likely to be abnormal) fits into each of the classes. The learning result from the first estimation model (the class estimated by the first estimation model) as overwritten by the learning result from the second estimation model (the class estimated by the second estimation model) is used as the final result. However, the embodiment is not limited to this, and the final result may be obtained by using both the first estimation model and the second estimation model. A description will now be given of such an embodiment (second embodiment).

A scheme of improving estimation capabilities in response to data not learned yet by combining learning results from different estimation models (ensemble learning) is known in machine learning. Using this scheme can correct variance (variation in estimated values) occurring in individual learning models and avoid resultant overlearning. The second embodiment utilizes this teaching.

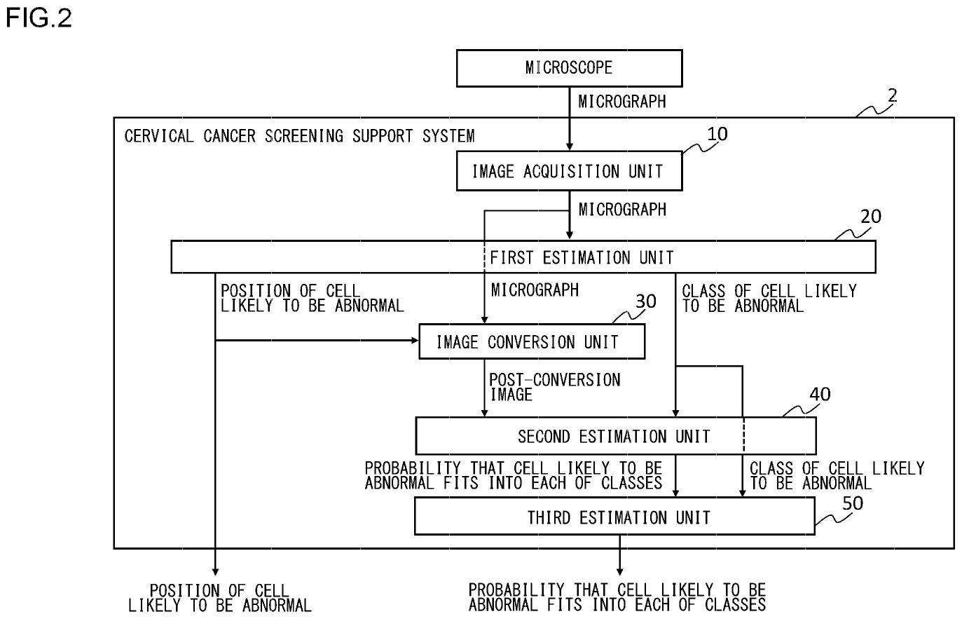

FIG. 2 is a functional block diagram of a cervical cancer screening support system 2 according to the second embodiment. The cervical cancer screening support system 2 is provided with an image acquisition unit 10, a first estimation unit 20, an image conversion unit 30, a second estimation unit 40, and a third estimation unit 50. In other words, the cervical cancer screening support system 2 is provided with the third estimation unit 50 in addition to the features of the cervical cancer screening support system 1 of FIG. 1. The rest of the configuration of the cervical cancer screening support system 2 is common to the cervical cancer screening support system 1. The third estimation unit 50 will be described hereinafter, and a description of the common configuration will be omitted.

When the class of the cell likely to be abnormal is input from the first estimation unit and the probability that the cell likely to be abnormal fits into each of the classes is input from the second estimation unit, the third estimation unit 50 uses the third estimation model to estimate and output the probability that the cell likely to be abnormal fits into each of the classes. The third estimation model is generated by integrating the class estimated by using the first estimation model as being applicable to the cell likely to be abnormal and the probability, estimated by using the second estimation model, that the cell in the post-conversion image fits into each of the classes. The third estimation model estimates the probability that the cell likely to be abnormal fits into each of the classes. It is preferable to use, for example, stacking ensemble learning (see, for example, non-patent literature 5) from the perspective of excellent capabilities to improve estimation precision. However, the embodiment is not limited to this, and suitable, arbitrary ensemble learning may be used.

In the embodiments described above, the image acquisition unit may be provided in a smartphone. Further, the first estimation unit, the image conversion unit, and the second estimation unit may be provided in a data processing apparatus connected to the smartphone via a network. In this case, the image acquisition unit is typically a camera on a smartphone. Further, the data processing apparatus is, for example, a server or a cloud server installed in a data center. The network connecting the smartphone and the data processing apparatus may be any of a wired network, a wireless network, the Internet, an intranet, a public circuit, a leased line, etc. From the perspective of high-speed transmission of image data and convenience, a high-speed mobile communication network such as 5G is favorable.

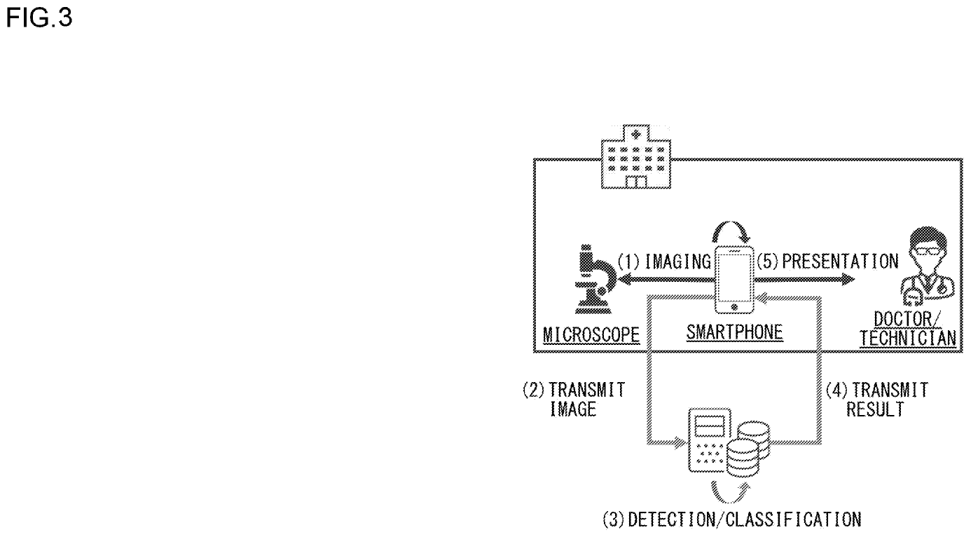

FIG. 3 schematically shows the cervical cancer screening support system of this embodiment. The camera on a smartphone takes a micrograph of a cell subject to examination (1). The image of the microscopic image thus taken is transmitted to the data processing apparatus at a remote location via the network (2). The data processing apparatus refers to the received microscopic image to estimate the position of the cell likely to be abnormal and the probability that the cell likely to be abnormal fits into each of the classes (3) and transmits the estimation result to the smartphone via the aforementioned network (4). The aforementioned estimation result received by the smartphone is presented to a doctor or a cytotechnologist and supports their diagnosis (5).

By building the cervical cancer screening support system in this way, a micrograph in medical practice can be taken by a smartphone, which is compact and easily available. Meanwhile, machine learning and screening processes, which require computer resources, can be performed by the high-performance data processing apparatus. Therefore, suitable functionality sharing can be realized.

In the embodiments described above, an external apparatus may be connected to the data processing apparatus via a network. By building the cervical cancer screening support system in this way, it is possible, for example, to share the system between apparatuses in multiple medical institutions.

The data processing apparatus in the embodiments described above may be provided with a database that stores the position of the cell likely to be abnormal and the class applicable to the cell likely to be abnormal estimated by the first estimation unit, and the probability, estimated by the second estimation unit, that the cell in the post-conversion image fits into each of the classes. By building the cervical cancer screening support system in this way, the result of machine learning can be turned into a database so that the usefulness is improved.

In the embodiments described above, the cervical cancer screening support system may be connected via a network to an external system provided with an equivalent cervical cancer screening support system. The database of the main system may store the position of the cell likely to be abnormal and the class applicable to the cell likely to be abnormal estimated by the first estimation unit of the external system, and the probability, estimated by the second estimation unit of the external system, that the cell in the post-conversion image fits into each of the classes.

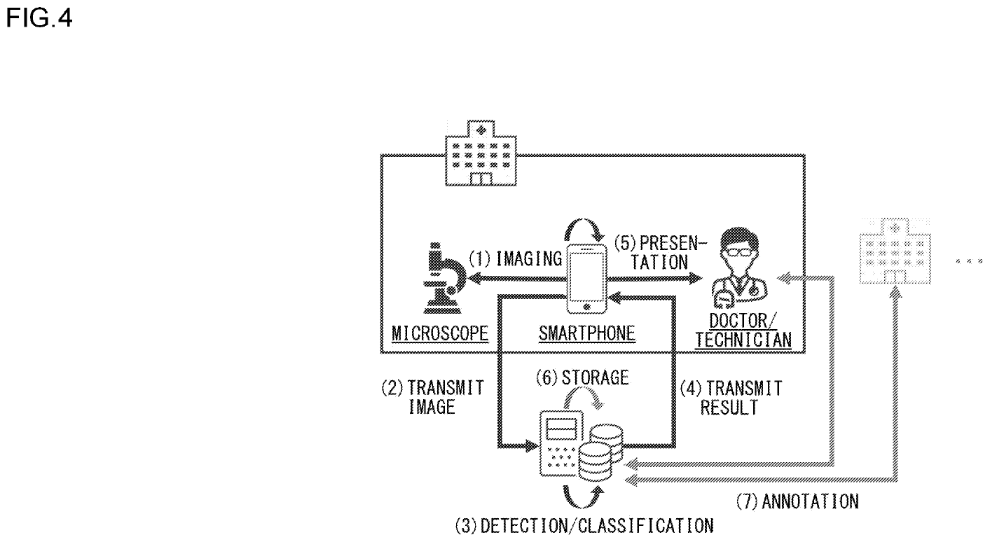

FIG. 4 schematically shows a cervical cancer screening support system of this embodiment. The camera on a smartphone takes a micrograph of a cell subject to examination (1). The microscopic image thus taken is transmitted to the data processing apparatus at a remote location via the network (2). The data processing apparatus refers to the received microscopic image to estimate the position of the cell likely to be abnormal and the probability that the cell likely to be abnormal fits into each of the classes (3) and transmits the estimation result to the smartphone via the aforementioned network (4). The aforementioned estimation result received by the smartphone is presented to a doctor or a cytotechnologist and supports their diagnosis (5). The database stores the position of the cell likely to be abnormal and the class applicable to the cell likely to be abnormal estimated by the first estimation unit, and the probability, estimated by the second estimation unit, that the cell in the post-conversion image fits into each of the classes (6). Meanwhile, machine learning is also performed in the external system connected via the network to the main system. The database of the main system also stores the position of the cell likely to be abnormal and the class applicable to the cell likely to be abnormal estimated by the first estimation unit of the external system, and the probability, estimated by the second estimation unit of the external system, that the cell in the post-conversion image fits into each of the classes (7).

By building the cervical cancer screening support system in this way, the results of machine learning performed in multiple systems can be aggregated so that an estimation model can be generated at a high speed and high precision.

### Third Embodiment

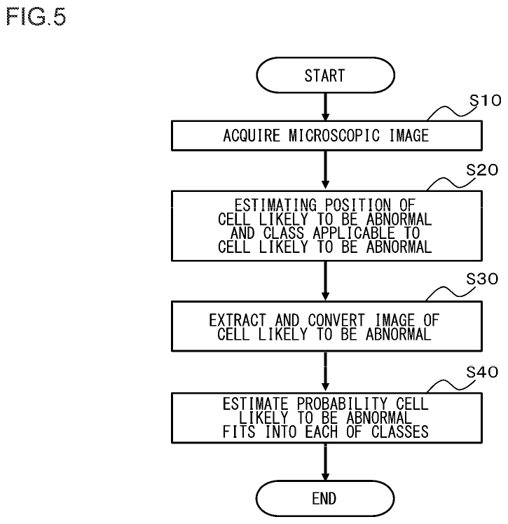

FIG. 5 is a flowchart showing steps in a cervical cancer screening support method according to the third embodiment. The method includes step S10 of acquiring a microscopic image, step S20 of estimating the position of a cell likely to be abnormal and a class applicable to the cell likely to be abnormal, step S30 of extracting and converting an image of the cell likely to be abnormal, and step S40 of estimating a probability that the cell likely to be abnormal fits into each of the classes.

In step S10, the method acquires a micrograph of a cell collected from the cervix of uterus by using an image acquisition unit.

In step S20, the method estimates, when the micrograph acquired by the image acquisition unit is input, the position of the cell found to be likely to be abnormal in the micrograph and the class applicable to the cell likely to be abnormal, by using a first estimation model generated through machine learning according to an object detection algorithm, using, as training data, the micrograph, the position of the cell found to be likely to be abnormal in the micrograph, and the class applicable to each cell likely to be abnormal.

In step S30, the method extracts from the micrograph an image of each cell located at the position estimated in the first step for estimation and converts each extracted image into a post-conversion image of a predetermined format.

In step S40, the method estimates, when the post-conversion image is input, the probability that the cell in the post-conversion image fits into each of the classes, by using a second estimation model generated through machine learning according to an image classification algorithm, using, as training data, the image of the cell likely to be abnormal and the probability that the cell likely to be abnormal fits into each of the classes.

According to this method, it is possible to estimate the position of a cell likely to be abnormal and the probability that the cell likely to be abnormal fits into each of the classes with high precision in cytodiagnosis for uterine cervical cancer screening. This improves precision of diagnosis and reduces loads on specialist doctors and cytotechnologists.

### Fourth Embodiment

A computer program according to the fourth embodiment causes a computer to execute the processing flow of FIG. 5. In other words, the program causes a computer to execute step S10 of acquiring a microscopic image; step S20 of estimating a position of a cell likely to be abnormal and a class applicable to the cell likely to be abnormal; step S30 of extracting and converting an image of the cell likely to be abnormal; and step S40 of estimating a probability that the cell likely to be abnormal fits into each of the classes.

According to this embodiment, the cervical cancer screening support program can be implemented on software so that screening of cervical cancer can be supported with high speed and high precision by using a computer.

### Fifth Embodiment

A smartphone application according to the fifth embodiment includes the cervical cancer screening support program described above.

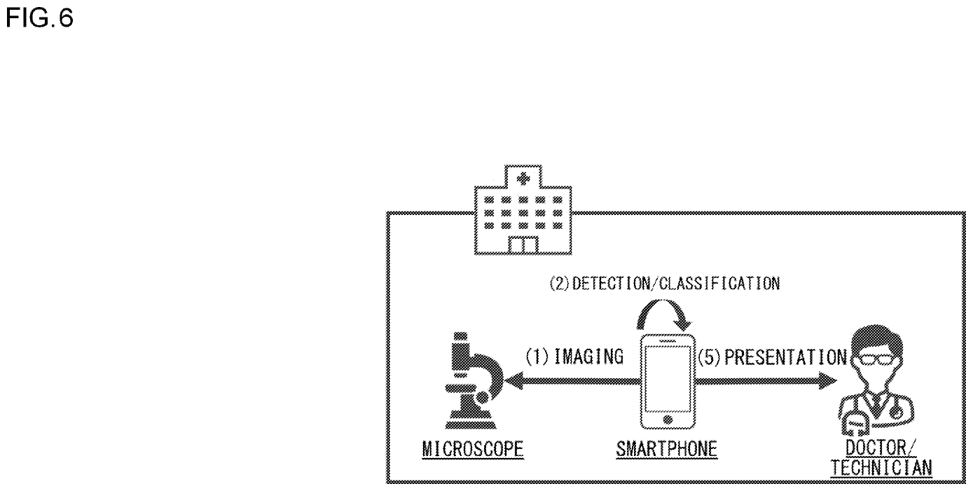

FIG. 6 schematically shows an embodiment of the smartphone application.

By realizing the cervical cancer screening support program as a smartphone application, it is possible to support screening of cervical cancer by using a smartphone, which is compact and easily available.

Hereinafter, a set of cells bindable to each other will be referred to as a “cell aggregate” in this specification. Also, the term “cells” in this specification encompasses “isolated and dispersed cells” and “cell aggregate”. To address numerous cases, research and development of cytodiagnosis support apparatuses (automated cervical cancer screening apparatuses) for diagnosis of cervix of uterus have been pursued. Liquid-based cytologic study (hereinafter, “LBC”) has been one of important steps in cytodiagnosis support apparatuses. In this case, an improved pre-process for minimizing overlapping of cells has been required to allow an automated apparatus to better discriminate abnormal cells. In this background, an approach that focuses on a pre-diagnosis cell treatment method directed to “avoiding formation of cell aggregate and cell overlapping” has been adopted in related-art cytodiagnosis support apparatuses. When there is marked cell overlapping or cytolysis, for example, it may be difficult to estimate the number of cells in squamous epithelium. This is because it is difficult to evaluate individual cell forms when cells aggregate. In this background, automatic diagnosis support apparatuses developed in the related art have been built to focus on a determination on single-cell basis using LBC by avoiding overlapping of cells and dispersing cells relatively finely.

Meanwhile, there are also cases where features unique to respective cell aggregates are identified. For example, unlike an endometrial cell sphere, a HSIL cell aggregate is not normally spherical, and the boundary thereof is not in an orderly shape. Further, in intraepithelial adenocarcinoma, a palisading or plumate aggregate is formed, and an image showing nuclear projection is identified. Still further, atypical cells form conglomeration in association with a change in intercellular junction more often than normal cells. In the field of urinology, too, a large-scale cellular population observed in natural urine is first considered as an indication of a tumorous lesion. Thus, cells when observed may sometimes present formation of characteristic agglomeration depending on the atypia or type. The tendency as described above inspired us to consider a possibility of increasing precision of evaluation by positively exploiting the presence of a cell aggregate for decision on atypia contrary to the approach of “avoiding formation of cell aggregate and cell overlapping” in the related art.

Cell aggregates are found in a variety of forms, and it is virtually impossible to input their features manually to the diagnosis support system. Specifically, agglomeration and overlapping form planes or 3D structures in infinite variation on large and small scales and are not as simple as single-cell nuclear structures on which the current distribution systems are built. Faced with these challenges, we have proposed “automatic extraction of features from cell aggregate images through deep learning” and have been able to obtain favorable results. More specifically, we have realized a cervical cancer screening support system that realizes highly precise screening by causing marked cell aggregates including atypical cells to be deeply learned as training data along with atypia classes of cells included in the cell aggregates.

### Sixth Embodiment

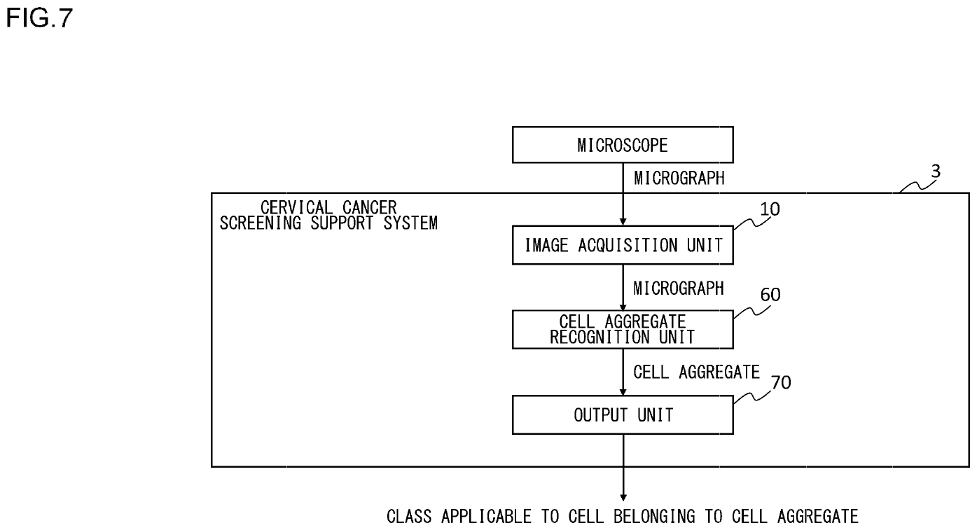

FIG. 7 is a functional block diagram of a cervical cancer screening support system 3 according to the sixth embodiment. The cervical cancer screening support system 3 is provided with an image acquisition unit 10, a cell aggregate recognition unit 60, and an output unit 70.

The image acquisition unit 10 acquires a micrograph of a cell collected from the cervix of uterus. The cell aggregate recognition unit 60 recognizes a cell aggregate in the micrograph acquired. For recognition of a cell aggregate, optional machine learning or deep learning may be used. The output unit 70 outputs a class to a cell belonging to the cell aggregate.

According to this embodiment, it is possible to positively exploit the presence of a cell aggregate for decision on atypia and to improve precision of diagnosis accordingly.

By way of one example, an area including the cell aggregate may include the background around the cell aggregate. In this case, the cell aggregate recognition unit 60 may use a frame having a predetermined shape to extract an image around the cell aggregate including the background. The frame may have an arbitrary shape such as a rectangle, square, arbitrary polygon, circle, and ellipse. According to this embodiment, information on not only the cell aggregate itself but also a necrotic substance, etc. included in the background can be included in the feature amount so that precision of diagnosis can be improved further.

By way of one example, the cell aggregate recognition unit may recognize a cell aggregate by using YOLO algorithm. Out study shows that YOLO algorithm is useful for recognition of cell aggregates. For this reason, precision of diagnosis can be improved further by using YOLO algorithm.

By way of one example, the cell aggregate recognition unit may recognize a cell aggregate in real time. According to this embodiment, a pre-process is not required so that efficiency of diagnosis can be improved further.

### Seventh Embodiment

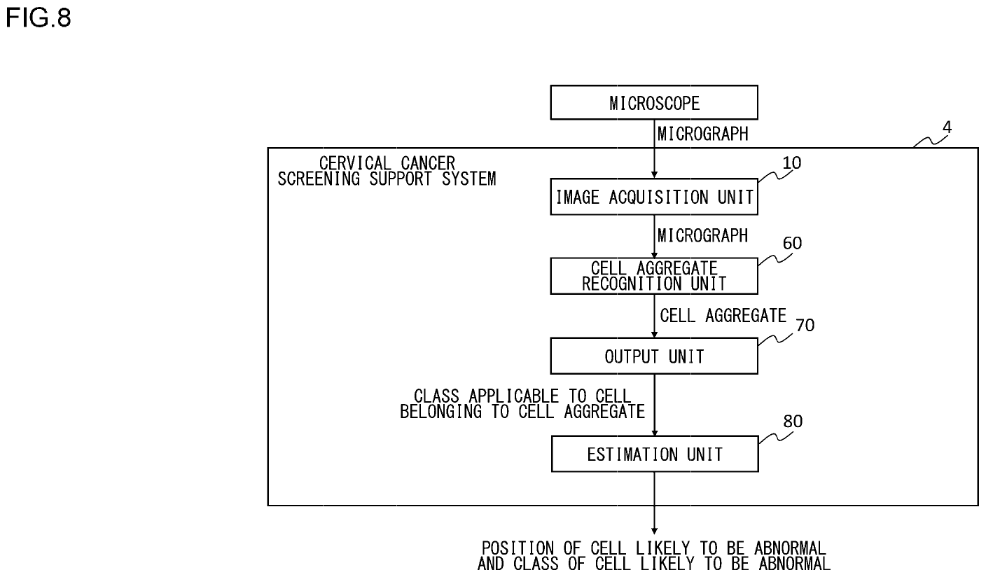

FIG. 8 is a functional block diagram of a cervical cancer screening support system 4 according to the seventh embodiment. The cervical cancer screening support system 4 is provided with an image acquisition unit 10, a cell aggregate recognition unit 60, an output unit 70, and an estimation unit 80. In other words, the cervical cancer screening support system 4 is provided with the estimation unit 80 in addition to the features of the cervical cancer screening support system 3 of FIG. 7. The rest of the configuration of the cervical cancer screening support system 4 is common to the cervical cancer screening support system 3.

The estimation unit 80 estimates and outputs, when a micrograph acquired by the image acquisition unit is input, a position of a cell found to be likely to be abnormal in the micrograph and a class applicable to the cell likely to be abnormal, by using an estimation model generated through machine learning according to an object detection algorithm, using, as training data, a marked cell aggregate, of cell aggregates recognized by the cell aggregate recognition unit, that includes an atypical cell and an atypia of a cell included in the marked cell aggregate.

More specifically, when a position of a cell aggregate recognized by the cell aggregate recognition unit 60 and an area including the cell aggregate are input, the estimation unit 80 uses the estimation model to estimate and output the position of a cell found to be likely to be abnormal and the class of the cell likely to be abnormal (i.e., which of predetermined classes that each of the cells likely to be abnormal fits into, the class being a result of examination for classification). For example, a case is considered where the estimation model determines that there are 10 cells likely to be abnormal and estimates the positions of these 10 cells. In this process, the estimation unit 80 calculates, as position information on each of the 10 cells, the x coordinate, y coordinate, height, and width of a rectangular area in which the cell is included in a floating decimal mode. In this process, the estimation model estimates which of ASC-US, LSIL, ASC-H, HSIL, and SCC of Bethesda classification mentioned above is applicable to each of the 10 cells (NILM means “not abnormal” and so is excluded). The estimation unit 80 calculates, as class information for each of the 10 cells, an integer value for discriminating a Bethesda class applicable to the cell. Thus, the estimation unit 80 outputs the position of the cell likely to be abnormal and the class of the cell likely to be abnormal.

The estimation model is generated through machine learning according to an object detection algorithm, using, as training data, a micrograph including a cell aggregate, a position of the cell aggregate and an area including the cell aggregate, and a marked cell aggregate, of cell aggregates recognized by the cell aggregate recognition unit, that includes an atypical cell. In the case of a cell likely to be abnormal such as a tumor, in particular, the characteristics thereof such as shape, size, color, chromatic value, gray scale, texture, and inversion identified in the image are different from those of healthy cells. These characteristics identified in the image can be highlighted depending on the case by subjecting raw image data to a process such as image inversion (horizontal and vertical), blurring, noise cancellation, gamma correction, and filtering. It is therefore possible to estimate the position of cell likely to be abnormal and the class applicable thereto with high precision, by using the micrograph including these features as training data and using an appropriate object detection algorithm.

According to this embodiment, it is possible to estimate the position of a cell likely to be abnormal and a class applicable to the cell likely to be abnormal with high precision because the presence of a cell aggregate can be positively exploited for decision on atypia in cytodiagnosis for uterine cervical cancer screening.

### Eighth Embodiment

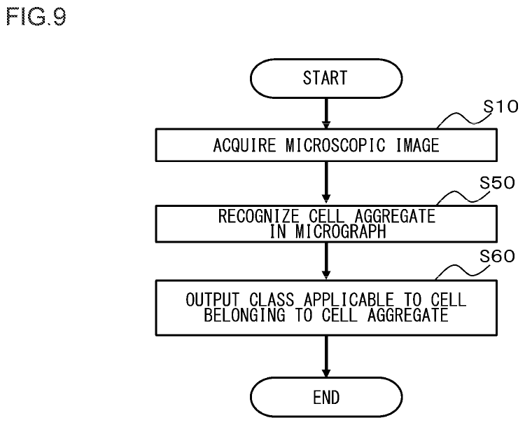

FIG. 9 is a flowchart showing steps in a cervical cancer screening support method according to the eighth embodiment. The method includes step S10 of acquiring, by using an image acquisition unit, a microscopic image, step S50 of recognizing a cell aggregate in the micrograph, and step S60 of outputting a class applicable to a cell belonging to the cell aggregate.

In step S10, the method acquires a micrograph of a cell collected from the cervix of uterus by using an image acquisition unit.

In step S50, the method recognizes a cell aggregate in the micrograph acquired in step S10. For recognition of a cell aggregate, optional machine learning or deep learning may be used.

In step S60, the method outputs a class applicable to a cell belonging to the cell aggregate.

According to this method, it is possible to positively exploit the presence of a cell aggregate for decision on atypia and to improve precision of diagnosis accordingly.

### Ninth Embodiment

A computer program according to the ninth embodiment causes a computer to execute the processing flow of FIG. 9. In other words, the program causes a computer to execute step S10 of acquiring a microscopic image; cell aggregate recognition step S50 of recognizing a cell aggregate in the micrograph; and output step S60 of outputting a class applicable to a cell belonging to the cell aggregate.

According to this embodiment, the cervical cancer screening support program can be implemented on software so that screening of cervical cancer positively exploiting the presence of a cell aggregate for decision on atypia can be supported by using a computer.

The present invention has been described above based on an embodiment. The embodiment is intended to be illustrative only and it will be understood by those skilled in the art that various modifications to combinations of constituting elements and processes are possible and that such modifications are also within the scope of the present invention.

1. A cervical cancer screening support system comprising:
an image acquisition unit that acquires a micrograph of a cellular specimen for cytodiagnosis of a cervix of uterus;
a cell aggregate recognition unit that recognizes a cell aggregate in the micrograph for atypia classification based on a cell aggregate in the cellular specimen; and
an output unit that outputs a class, including an atypia, applicable to a cell belonging to the cell aggregate.

2. The cervical cancer screening support system according to claim 1, wherein the cell aggregation recognition unit performs LBC on the cellular specimen collected.

3. The cervical cancer screening support system according to claim 1, wherein
the output unit outputs a class, including an atypia, by using deep learning to automatically extract a feature depending on the atypia or type from the cell aggregate.

4. The cervical cancer screening support system according to claim 1, wherein
an area including the cell aggregate includes a background around the cell aggregate.

5. The cervical cancer screening support system according to claim 1, wherein
the cell aggregate recognition unit recognizes a cell aggregate by using YOLO algorithm.

6. The cervical cancer screening support system according to claim 1, wherein
the cell aggregate recognition unit recognizes a cell aggregate in real time.

7. A cervical cancer screening support system according to claim 1, further comprising:
an estimation unit that estimates and outputs, when a micrograph acquired by the image acquisition unit is input, a position of a cell found to be likely to be abnormal in the micrograph and a class applicable to the cell likely to be abnormal, by using an estimation model generated through machine learning according to an object detection algorithm, using, as training data, a marked cell aggregate, of cell aggregates recognized by the cell aggregate recognition unit, that includes an atypical cell and an atypia of a cell included in the marked cell aggregate.

8. A cervical cancer screening support system comprising:
an image acquisition unit that acquires a micrograph of a cell collected from a cervix of uterus;
a first estimation unit that estimates and outputs, when the micrograph acquired by the image acquisition unit is input, a position of a cell found to be likely to be abnormal in the micrograph and a class applicable to the cell likely to be abnormal, by using a first estimation model generated through machine learning according to an object detection algorithm, using, as training data, the micrograph, the position of the cell found to be likely to be abnormal in the micrograph, and the class applicable to the cell likely to be abnormal, the class being a result of examination for classification; and
an image conversion unit that extracts from the micrograph an image of each cell located at the position estimated by the first estimation unit and converts each extracted image into a post-conversion image of a predetermined format; and
a second estimation model that estimates and outputs, when the post-conversion image is input, a probability that the cell in the post-conversion image fits into each of the classes by using a second estimation model generated through machine learning according to an image classification algorithm, using, as training data, the image of the cell likely to be abnormal and the probability that the cell likely to be abnormal fits into each of the classes.

9. The cervical cancer screening support system according to claim 8, wherein
the class is a class of Bethesda classification.

10. The cervical cancer screening support system according to claim 8, wherein
the object detection algorithm is YOLO algorithm.

11. The cervical cancer screening support system according to claim 8, wherein
the image classification algorithm is a convolutional neural network.

12. The cervical cancer screening support system according to claim 8, further comprising:
a third estimation model generated by integrating the class estimated by the first estimation model as being applicable to the cell likely to be abnormal and the probability, estimated by the second estimation model, that the cell in the post-conversion image fits into each of the classes, the third estimation model estimating and outputting the probability that the cell likely to be abnormal fits into each of the classes.

13. The cervical cancer screening support system according to claim 12, wherein
the third estimation model is generated by using stacking ensemble learning.

14. The cervical cancer screening support system according to claim 8, comprising:
a smartphone including the image acquisition unit; and
a data processing apparatus connected to the smartphone via a network and including the first estimation unit, the image conversion unit, and the second estimation unit.

15. The cervical cancer screening support system according to claim 14, wherein
an external apparatus is adapted to be connected to the data processing apparatus.

16. The cervical cancer screening support system according to claim 14, wherein
the data processing apparatus includes a database that stores the position of the cell likely to be abnormal and the class applicable to the cell likely to be abnormal estimated by the first estimation unit, and the probability, estimated by the second estimation unit, that the cell in the post-conversion image fits into each of the classes.

17. The cervical cancer screening support system according to claim 16, wherein
the cervical cancer screening support system is connected via a network to an external network including the cervical cancer screening support system, wherein
the database stores the position of the cell likely to be abnormal and the class applicable to the cell likely to be abnormal estimated by the first estimation unit of the external system, and the probability, estimated by the second estimation unit of the external system, that the cell in the post-conversion image fits into each of the classes.

18. The cervical cancer screening support system according to claim 8, comprising a cell aggregate recognition unit that recognizes a cell aggregate in the micrograph for atypia classification based on a cell aggregate in a cellular specimen.

19. A cervical cancer screening support method comprising:
acquiring, by using an image acquisition unit, a micrograph of a cellular specimen for cytodiagnosis of a cervix of uterus;
recognizing a cell aggregate in the micrograph for atypia classification based on a cell aggregate in the cellular specimen; and
outputting a class, including an atypia, applicable to a cell belonging to the cell aggregate.

20. The cervical cancer screening support method according to claim 19, further comprising:
performing LBC on the cellular specimen collected.

21. The cervical cancer screening support method according to claim 19, wherein
the outputting includes outputting a class, including an atypia, by using deep learning to automatically extract a feature depending on the atypia or type from the cell aggregate.

22. A cervical cancer screening support method comprising:
acquiring, by using an image acquisition unit, a micrograph of a cell collected from a cervix of uterus;
a first estimation estimating, when the micrograph acquired by the image acquisition unit is input, a position of a cell found to be likely to be abnormal in the micrograph and a class applicable to the cell likely to be abnormal, by using a first estimation model generated through machine learning according to an object detection algorithm, using, as training data, the micrograph, the position of the cell found to be likely to be abnormal in the micrograph, and the class applicable to the cell likely to be abnormal, the class being a result of examination for classification; and
extracting from the micrograph an image of each cell located at the position estimated by the first estimation and converting each extracted image into a post-conversion image of a predetermined format; and
a second estimation estimating, when the post-conversion image is input, a probability that the cell in the post-conversion image fits into each of the classes by using a second estimation model generated through machine learning according to an image classification algorithm, using, as training data, the image of the cell likely to be abnormal and the probability that the cell likely to be abnormal fits into each of the classes.

23. A recording medium encoded with a cervical cancer screening support program for causing a computer to execute a method comprising:
acquiring, by using an image acquisition unit, a micrograph of a cellular specimen for cytodiagnosis of a cervix of uterus;
recognizing a cell aggregate in the micrograph for atypia classification based on a cell aggregate in the cellular specimen; and
outputting a class, including an atypia, applicable to a cell belonging to the cell aggregate.

24. The recording medium according to claim 23 encoded with a cervical cancer screening support program, the method further comprising:
performing LBC on the cellular specimen collected.

25. The recording medium according to claim 23 encoded with a cervical cancer screening support program, wherein
the outputting outputs a class, including an atypia, by using deep learning to automatically extract a feature depending on the atypia or type from the cell aggregate.

26. A recording medium encoded with a cervical cancer screening support program for causing a computer to execute a method comprising:
acquiring, by using an image acquisition unit, a micrograph of a cell collected from a cervix of uterus;
a first estimation estimating, when the micrograph acquired by the image acquisition unit is input, a position of a cell found to be likely to be abnormal in the micrograph and a class applicable to the cell likely to be abnormal, by using a first estimation model generated through machine learning according to an object detection algorithm, using, as training data, the micrograph, the position of the cell found to be likely to be abnormal in the micrograph, and the class applicable to the cell likely to be abnormal, the class being a result of examination for classification;
extracting from the micrograph an image of each cell located at the position estimated by the first estimation and converting each extracted image into a post-conversion image of a predetermined format; and
a second estimation estimating, when the post-conversion image is input, a probability that the cell in the post-conversion image fits into each of the classes by using a second estimation model generated through machine learning according to an image classification algorithm, using, as training data, the image of the cell likely to be abnormal and the probability that the cell likely to be abnormal fits into each of the classes.

27. A smartphone having a smartphone application installed therein, the smartphone application including the cervical cancer screening support program according to claim 23.
